SEQUENCE LISTING (10 sequences)

SEQ ID NO: 1 - DNA, 23 bp, Homo sapiens
  source 1..23: mol_type = genomic DNA
tccactcggataagatgctgagg

SEQ ID NO: 2 - protein, 8 aa, Homo sapiens
EVRILHQP

SEQ ID NO: 3 - DNA, 23 bp, synthetic construct
  source 1..23: mol_type = other DNA
tccattcggataagatgctgagg

SEQ ID NO: 4 - protein, 8 aa, synthetic construct
EMRILHQP

SEQ ID NO: 5 - DNA, 23 bp, synthetic construct
  source 1..23: mol_type = other DNA
tccacttggataagatgctgagg

SEQ ID NO: 6 - protein, 8 aa, synthetic construct
EVQILHQP

SEQ ID NO: 7 - DNA, 23 bp, synthetic construct
  source 1..23: mol_type = other DNA
tccatttggataagatgctgagg

SEQ ID NO: 8 - protein, 8 aa, synthetic construct
EMQILHQP

SEQ ID NO: 9 - DNA, 23 bp, synthetic construct
  source 1..23: mol_type = other DNA
tccagttggataagatgctgagg

SEQ ID NO: 10 - protein, 8 aa, synthetic construct
ELQILHQP

(19) United States
(12) Patent Application Publication
(10) Pub. No.: US 2026/0193642 A1
(43) Pub. Date: Jul. 9, 2026

(54) DIRECT MEASUREMENT OF ENGINEERED CANCER MUTATIONS AND THEIR TRANSCRIPTIONAL PHENOTYPES IN SINGLE CELLS

(71) Applicant: The Board of Trustees of the Leland Stanford Junior University, Stanford, CA (US)

(72) Inventors: Hanlee P. JI, Stanford, CA (US); Heonseok KIM, Palo Alto, CA (US)

(21) Appl. No.: 19/124,249

(22) PCT Filed: Oct. 26, 2023
(86) PCT No.: PCT/US2023/077947

§ 371 Date: Apr. 24, 2025

Related U.S. Application Data
(60) Provisional application No. 63/420,047, filed on Oct. 27, 2022.

(51) Int. Cl. C12N 15/10

(52) U.S. Cl. CPC C12N 15/1082; C12N 15/1065

(57) ABSTRACT

Provided herein is a method for analyzing cells. In some embodiments the method may comprise base editing a target gene in a population of cells to produce genetically modified cells, reverse transcribing mRNA from single cells in the population of cells to produce cDNA, wherein the cDNA produced by each cell has a cell barcode and a unique molecular identifier (UMI), amplifying and sequencing cDNA transcribed from the target gene, to determine the identity of the edited base, on a cell-by-cell basis, performing gene expression analysis on a cell-by-cell basis using short-read sequencing and comparing the results for each cell, to determine how the edited base alters gene expression.

### CROSS-REFERENCING

This application claims the benefit of provisional application Ser. No. 63/420,047, filed on Oct. 27, 2022, which application is incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, “STAN-2045WO_SEQ_LIST”, created on Oct. 23, 2023, and having a size of 9,809 bytes. The contents of the Sequence Listing XML are incorporated herein by reference in their entirety.

### BACKGROUND

Ongoing genomic studies of cancer are cataloguing extensive numbers of somatic variants. For example, genome sequencing studies have identified numerous cancer mutations across a wide spectrum of tumor types. Many of these mutations result in amino acid substitutions. Given the sheer number of discovered mutations, determining the phenotype of cancer substitutions with functional characterization remains an enormous challenge. In-silico functional predictions of cancer mutations are frequently used as a solution. However, these computational methods do not provide more discrete biological characterization. There remains a significant need for high throughput approaches to functionally evaluate many mutations in an efficient manner. CRISPR base editors and single guide RNAs (sgRNAs) have been used for genetic screens, where they directly introduce specific variants into target genes at their native genomic loci among transduced cells. Studies using this method examined the altered cellular fitness resulting from the introduced genetic variants, either by counting sgRNA or barcode sequences among the cell pool, however these approaches do not directly verify the presence of an engineered mutation since the association with a genotype is imputed based on the sgRNA or the barcode sequence.

Base editors can introduce multiple variants into a target genomic sequence. Although a given sgRNA sequence is intended to generate a single variant, the actual base editing process introduces multiple different, unintended variants at the target genomic sequence. For example, when using the cytosine base editor (CBE), the conversion of the either a C to T or a C to G produces different variants other than what was intended. CBEs exhibit cytosine editing in both the target and neighboring bystander cytosines in the editing window with the outcome being multiple different variants at the target sequence site. This variability points to the need to directly genotype the base editor target site as the best approach for verifying the intended mutation being present. Direct validation of an engineered mutation is a necessary step if one is to accurately determine the phenotype, and this requires examining individual cells.

Some studies have employed a reporter system to infer the presence of engineered mutations, but this is an indirect approach and assumes the same genome edit has occurred in both the reporter and endogenous site. Also, these methods may not reflect the precise effects of mutations on gene expression. For example, the single-cell Perturb-seq method was adapted to exogenously express genes in the form of cDNAs containing a specific variant, and then indirectly measure the mutated gene using a barcode sequence (Ursu, et al 2002). Although one can interrogate the resultant single-cell transcriptome changes induced by each variant, this approach has limitations. Specifically, the gene variant is expressed with an exogenous promoter which is not under canonical genetic regulation at the gene's native locus. Second, variants are delivered to cells with wild-type gene expression of the target gene, which can mask the effect of the variant on protein function. Third. only the barcode sequence is detected instead of the variant itself. Moreover, template switching in lentivirus packaging can induce swapping of the variant-barcode association, leading to artifacts in identification and transcriptional phenotyping.

The present method is believed to addresses these issues. This method is referred to as transcript-informed single-cell CRISPR sequencing (TISCC-Seq).

### SUMMARY

In some embodiments, the present method comprising: (a) base editing a target gene in a population of cells to produce genetically modified cells; (b) reverse transcribing mRNA from single cells in the population of cells to produce cDNA, wherein the cDNA produced by each cell has a cell barcode and a unique molecular identifier (UMI); (c) amplifying and sequencing cDNA transcribed from the target gene, to determine the identity of the edited base, on a cell-by-cell basis; (d) performing gene expression on a cell-by-cell basis using short-read sequencing; and (e) comparing the results of (c) and (d) for each cell, to determine how the edited base alters gene expression. In some embodiments, (c) is done by long-range sequencing, the long-read sequencing comprises single molecule real time (SMRT) sequencing or nanopore sequencing. In some embodiments, (b) may be done by encapsulating each cell in a droplets and creating the cDNA in the droplets, although other methods are possible. In some embodiments, step (d) may be done by short range sequencing (e.g., reversible terminator sequencing). Any embodiment may comprise contacting the genetically modified cells with a drug candidate to determine whether the candidate reverses any changes in gene expression that are caused by the edited base.

Depending on how the method is implemented, the method may rely on a CRISPR base editor to introduce multiple endogenous genetic variants into a given genomic target. Long-read sequencing identifies these mutations directly from a target's transcript sequence at single-cell resolution. Then, the short-read transcriptome profile is integrated from the same single cells. This integrative approach can enable single-cell direct genotyping and phenotyping of various genetic variants introduced into the native gene locus. Single-cell characterization allows one to distinguish the base editor's intended versus unintended mutations among individual cells.

These and other aspects and advantages will become apparent in view of the description that follows below.

### BRIEF DESCRIPTION OF THE DRAWINGS

The skilled artisan will understand that the drawings, described below, are for illustration purposes only. The drawings are not intended to limit the scope of the present teachings in any way.

FIGS. 1A-1C. Schematic of TISCC-seq. (FIG. 1A) Overview of direct detection and phenotyping of various TP53 coding mutations. (FIG. 1B) Schematic of the variant calling accuracy comparison between short- and long-read single-cell sequencing. (FIG. 1C) Accuracy of the mutation calling of long-read sequencing. Mutation sequences of each sgRNA target site were compared, and proportion of UMIs which have same sequence in short- and long-read sequencing was calculated.

FIGS. 2A-2D. TISCC-seq identifies mutations directly. (FIG. 2A) Overview of single-cell cDNA analysis pipe-line. (FIG. 2B) Structure of p53 protein and distribution of sgRNA target sites used in this study. TAD, transactivation domain; PRR, proline-rich region; OD, oligomerization domain; CTD, carboxyl terminus domain. (FIG. 2C) Dot plot showing the proportion of each genetic variant detected from single-cell cDNA and genomic DNA. Red dots represent variant with premature stop codon. (FIG. 2D) Cells with same sgRNA can result in various genotypes. The pie chart shows the proportion of resultant amino acid changes from cells with sgRNA targeting V197M mutation. Proportions of mutations are calculated from the single-cell cDNA long-read sequencing. Underlines indicate each triplet codon and number indicate position of the codon. Red DNA sequences indicate substituted bases and blues indicate PAM sequences. (WT, V197M, R196Q, R196Q_V197M, and R196Q_V197L nucleotide sequences correspond to SEQ ID NOs: 1, 3, 5, 7, and 9, respectively; WT, V197M, R196Q, R196Q_V197M, and R196Q_V197L amino acid sequences correspond to SEQ ID NOs: 2, 4, 6, 8, and 10, respectively).

FIGS. 3A-3G. TISCC-seq on HCT116 cells. (FIGS. 3A, 3B, 3C) UMAP plot showing single-cell gene expression profile per each genetic variant. HCT116 cells are treated with vehicle (FIG. 3A) or Nutlin-3a (FIG. 3B) after the introduction of variants using subset of sgRNA library. (FIG. 3C) HCT116 cells are treated with Nutlin-3a after introduction of variants using full sgRNA library. (FIG. 3D) Proportion of UMAP cluster from cells with each genetic variant. Hierarchical clustering was performed based on the proportion to categorize genetic variants. Reds indicate wild type-like variants. (FIG. 3E) UMAP embedding of cells colored by p53 pathway gene scores. (FIG. 3F) Violin plot showing p53 pathway gene score per cells with each genetic variant. *: P<0.03, n.s: Not significant; two-sided t-test. P=1.7e-33, 3.7e-29, 1.3e-06, 2.1e-14, 1.5e-06, 3.8e-07, 2.1e-02, 9.5e-09, 7.8e-05, 2.6e-07, 7.2e-27, 6.9e-04, 3.9e-07, 1.5e-88, 2.8c-06, 2.0c-30, 8.7e-23, 5.0e-67, 1.4e-09, 4.4e-14, 5.7e-14, 3.3c-37, 3.0e-13, 5.8e-38, 1.5e-10, 1.5e-43, 7.5e-04, 8.6e-09, 5.5e-05, 4.3e-23, 3.1e-07, 9.2e-03, 1.2e-03, 1.4e-05, 1.3e-05, 6.3e-04, 2.3e-12, 8.6e-65, 7.2e-41, 1.1e-10, 1.8e-49, 2.1e-25, 3.8e-04, 7.2e-35, 4.2e-20, 2.0e-04, 5.0e-35, 2.0e-50, 8.0e-23, 8.9e-43, 1.4e-52, 8.2e-42, 4.2e-29, 3.8e-21, 1.8e-31, 1.7e-47, 7.3e-08, 2.2e-34, 8.7e-31, 2.2e-45, 6.1e-08, 8.2e-06, 7.6e-40, 7.0e-14, 5.7e-10, 2.1e-25, 8.6e-32, 5.3e-05, 5.3e-01, 5.7e-01, 4.6e-01, 2.6e-01, 3.7e-01, 3.8e-01. (FIG. 3G) Heatmap showing average GSVA enrichment score of selected Hallmark pathways per each category of genetic variant.

FIGS. 4A-4C. Confirmation of TISCC-seq. (FIGS. 4A, 4B) Heatmap showing the average GSVA enrichment score of selected Hallmark pathways. (FIG. 4A) Scores are calculated from single-cell analysis of heterogenous TP53 genetic variants pool. (FIG. 4B) Scores are calculated from bulk RNA sequencing from clonal cells with indicated TP53 genetic variants. (FIG. 4C) Cell cycle analysis using DNA content staining using clonal cells. Genetic variant per cells and nutlin-3a treatments are indicated. N=2 biologically independent cells. P<2.2e-16, P=0.95, 0.95. P values are calculated by Chi-squared test; two-sided.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a modified CRISPR/Cas effector polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

A “cell” as used herein, denotes an in vivo or in vitro eukaryotic cell or a cell line.

A “binding site for a guide-RNA” as used herein is a polynucleotide (e.g., DNA such as genomic DNA) that includes a site (“target site” or “target sequence”) targeted by a modified CRISPR/Cas effector polypeptide. The target sequence is the sequence to which the guide sequence of a guide nucleic acid (e.g., guide RNA; e.g., a dual guide RNA or a single-molecule guide RNA) will hybridize. For example, the target site (or target sequence) 5′-GAGCAUAUC-3′ within a target nucleic acid is targeted by (or is bound by, or hybridizes with, or is complementary to) the sequence 5′- -3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

As used herein, the term “long-read sequencing” refers to sequencing read lengths greater than 500 bases, particularly, longer than 600 bases. The term “short read sequencing” refers to sequencing read lengths less than 600 bases, particularly, less than 500 bases.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112. In describing and claiming the present invention, certain terminology will be used in accordance with the definitions set out below. It will be appreciated that the definitions provided herein are not intended to be mutually exclusive.

As used herein, the phrases “for example,” “for instance,” “such as,” or “including” are meant to introduce examples that further clarify more general subject matter. These examples are provided only as an aid for understanding the disclosure and are not meant to be limiting in any fashion.

As used herein, the terms “may,” “optional,” “optionally,” or “may optionally” mean that the subsequently described circumstance may or may not occur, so that the description includes instances where the circumstance occurs and instances where it does not.

Definitions of other terms and concepts appear throughout the detailed description.

### DESCRIPTION

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Other definitions of terms may appear throughout the specification.

In certain embodiments, the present method may comprise base editing a target gene in a population of cells to produce genetically modified cells. This step may comprise transfecting a population of cells en masse with appropriate materials (constructs), incubating the cells so that at least some of them contain nucleotide changes at one or more sites in a target gene, and then incubating the cells so that changes in gene expression can be observed. In some embodiments, the mutations may be clustered in a particular region of the gene. This step of the method may make cells that individually have one or two changes in the target gene, but collectively have at least 5 or at least 10 changes in the target gene.

The cells from any suitable organism, e.g., from bacteria, yeast, plants and animals, such as fish, birds, reptiles, amphibians and mammals may be used in the subject method. In certain embodiments, mammalian cells, i.e., cells from mice, rabbits, primates, or humans, or cultured derivatives thereof, may be used. The sample may contain cells that are in solution, e.g., cultured cells that have been grown as a cell suspension. In other embodiments, disassociated cells (which cells may have been produced by disassociating cultured cells or cells that are in a solid tissue, e.g., a soft tissue such as liver or spleen, etc. using trypsin or the like) may be used. In particular embodiments, the sample may contain blood cells, e.g., whole blood or a sub-population of cells thereof. Sub-populations of cells in whole blood include platelets, red blood cells (erythrocytes), platelets and white blood cells (i.e., peripheral blood leukocytes, which are made up of neutrophils, lymphocytes, eosinophils, basophils, and monocytes). The genome of these cells may be modified by the base editor.

Many mutations are single nucleotide variants, many of which lead to amino acid substitutions. Conventional CRISPR does not generate substitutions. Rather, CRISPR/Cas9 and other enzymes in the class introduces double stranded DNA breaks (DSBs)—this genomic alteration leads to insertions and deletions (indels). Given the general nature of the Cas9 break, other types of genomic alterations can be introduced such as large deletions and rearrangements. Base editors introduce point mutations without a DNA double-strand break (DSB) or a requirement for template donor DNA (Gaudelli Nature 2017 551, 464-471; Komor, Nature 533, 2016 420-424; Nishida, Science 2016 353:aaf8729; Kim, Nat Biotechnol. 2019 37:430-435). There are two general classes which include cytosine base editors (CBEs) and adenine base editors (ABEs). CBEs were developed by combining APOBEC1 enzymes, which remove an amine group from cytosine, with catalytically dead Cas9 (dCas9) or Cas9 nickase (nCas9) (Komor, 2016). ABEs involve fusing an adenine deaminase to the Cas9 variant. Because an adenine deaminase accepts single-stranded DNA as a substrate, researchers created new ssDNA-targetable enzymes with engineered adenine deaminases (Gaudelli, 2017; Kim, 2019, supra).

Based editors allow for engineering in specific point mutations into the genome and allows their detection at single cell resolution. It does this by using base editor technology to introduce the mutation followed by single cell long read sequencing to determine which cells have the mutation.

Next, the method may comprise reverse transcribing mRNA from the cells to produce cDNA, wherein the cDNA produced by each cell has a cell barcode and a unique molecular identifier (UMI). In these embodiments, the cells may be compartmentalized with beads that have primers (e.g., oligo(dT) primers that have an UMI (e.g., a random sequence) and a bead-specific sequence (a unique barcode for each bead) and, some embodiments, a PCR handle, such that some of the compartments contain a single cell and a single bead. The cells can be lysed to release RNA, which hybridizes to the primers and is revised transcribed. The resulting cDNA contains a bead-specific barcode (which becomes a cell-specific barcode) and a random sequence. After cDNA synthesis, the cDNA from the compartments may be pooled and sequenced en masse. The cell-specific barcodes allows one to identify sequence reads that originate from the same cell whereas the UMI allows one to count the numbers of starting molecules (even if they have the same sequence). These methods are described in a number of publications, including Zhang et al (Nature Communications 2020 11: 2118) and Delley et al (Scientific Reports 2011 1110857). In other embodiments, the cDNA may be made in sity and the single cell barcodes and UMIs may be added by an alternative method, such as a split-and-pool or drop-seq-based method, among others.

The next steps of the method may be performed in any order. Next, the method may comprise sequencing cDNA transcribed from the target gene, to determine the identity of the edited base on a cell-by-cell basis. In these embodiments, the method may comprise amplifying the transcript of the target gene in the cDNA in a way that the amplification product includes the cell-specific barcode. This may be done, e.g., using one gene-specific primer and a primer that recognizes the PCR handle, for example, although in some embodiments it is unnecessary to specifically amplify the target gene. In these latter embodiments, the cDNA may be sequenced directly, without amplifying the target gene first. The amplified cDNA can be sequenced, particularly, using long-read sequencing. In some cases, the long-read sequencing comprises single molecule real time (SMRT) sequencing or nanopore sequencing. The SMRT sequencing can be circular consensus sequencing or continuous long read sequencing.

Certain details of long-read sequencing, for example, SMRT (developed by Pacific Biosciences (PacBio)™) and nanopore sequencing (developed by Oxford Nanopore Technologies™) are described by the publication Logsdon et al. (2020), Long-read human genome sequencing and its applications, Nature Reviews Genetics, Vol. 21, pages 597-614, which is herein incorporated by reference in its entirety.

Briefly, in SMRT sequencing, an amplicon is ligated to hairpin adapters to form a circular molecule, called a SMRT bell. The SMRTbell is bound by a DNA polymerase and loaded onto a SMRT Cell for sequencing. A SMRT Cell can contain up to 8 million zero-mode waveguides (ZMWs). ZMWs are chambers of picolitre volumes. Light penetrates the lower 20-30 nm of SMRT Cells. The SMRTbell template and polymerase become immobilized on the bottom of the chamber. During the sequencing reaction, fluorescently labelled deoxynucleoside triphosphates (dNTPs) are incorporated into the newly synthesized strand, a fluorescent dNTP is held in the detection volume, and a light pulse from the well excites the fluorophore. A camera detects the light emitted from the excited fluorophore, which records the wavelength and the position of the incorporated base in the nascent strand. The DNA sequence is determined by the changing fluorescent emission that is recorded within each ZMW.

In nanopore sequencing, long DNA strand may be tagged with sequencing adapters preloaded with a motor protein on one or both ends. The DNA is combined with tethering proteins and loaded onto the flow cell for sequencing. The flow cell contains protein nanopores embedded in a synthetic membrane. The tethering proteins bring the molecules to be sequenced towards the nanopores and as the motor protein unwinds the DNA, an electric current is applied, which drives the negatively charged DNA through the pore. The DNA is sequenced as it passes through the pore and causes characteristic changes in the current. The amplification product may be sequenced using any suitable long range sequencing technology, e.g., nanopore sequencing (e.g., as described in Soni et al. Clin. Chem. 2007 53: 1996-2001, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477. See also Greninger Genome Medicine. 2015 1: 99, among others. The junction of the fusion can be identified in the sequence reads.

Long-read sequencing produces ‘long’ sequence reads of at least about 500 or at least about 600 bases. Particularly, long-read sequencing sequences at least 800, at least 1000, at least 1200, at least 1400, at least 1600, at least 1800, at least 2000, at least 2500, or at least 3,000 bases of the amplified products. Thus, the long-read sequence can be used to sequence a target mRNA of at least 500 to at least 3,000 bases in length.

Gene expression analysis on a cell-by-cell basis is performed using short-read sequencing. This may be done using any suitable scRNA-seq method. In these embodiments, the cDNA may be pooled, amplified, and sequenced. The amplification product may be sequenced by any suitable system including Illumina's reversible terminator method, Roche's pyrosequencing method (454), Life Technologies' sequencing by ligation (the SOLiD platform), Ultima Genomics (e.g. UG100™), singular genomics (e.g. G4 system), element biosciences (e.g. Aviti™ system), Life Technologies' Ion Torrent platform or Pacific Biosciences' fluorescent base-cleavage method Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps. The sequencing step may be done using any convenient next generation sequencing method and may result in at least 10,000, at least 100,000, at least 500,000, at least 1M at least 10M at least 100M, at least 1B or at least 10B sequence reads per reaction. In some cases, the reads may be paired-end reads. After sequencing, the sequence reads that have the same first index sequence or complement thereof and the same second index sequence or complement thereof may be grouped together. In these embodiments, the combination of the first index sequence or complement thereof and the second index sequence or complement thereof identifies a single biological particle (e.g., cell or nuclei) from a particular sample. Short read sequencing produces sequence reads on the range of 100 bases to 600 bases, e.g., 200-400 bases, which sequence reads may be paired end. As noted above, the cDNAs have been tagged with a random sequence and a cell-specific barcode, thereby allowing gene expression to be quantified on a transcript-by-transcript bases and a cell-by-cell basis. The reverse transcribing the transcriptomes can be performed using primers comprising: 1) random nucleotide sequences, for example, random hexamers, or 2) oligo-dT sequence. See, e.g., Trombetta et al (Curr Protoc Mol Biol. 2014 107: 1-4) among others. In some embodiments, the sequence reads may be analyzed to provide a quantitative determination of which sequences are in the sample. This may be done by, e.g., counting sequence reads or, alternatively, counting the number of original starting molecules, prior to amplification, based on their UMI sequence. Random barcodes and exemplary methods for counting individual molecules are described in Casbon (Nucl. Acids Res. 2011, 22 e81) and Fu et al (Proc Natl Acad Sci USA. 2011 108: 9026-31), among others. Molecular barcodes are described in US 2015/0044687, US 2015/0024950, US 2014/0227705, U.S. Pat. Nos. 8,835,358 and 7,537,897, as well as a variety of other publications.

In some embodiments, the method may comprise comparing the results of the long run sequencing and the short read sequencing for each cell, to determine how the edited base alters gene expression. As would be apparent, both datasets are barcoded in a cell-by-cell way such that the results obtained from the long range dataset can be linked to the results obtained from the short range dataset. In these embodiments, the identify of a base change in a target gene in a cell as well as a gene expression profile for the cell can be produced, for multiple cells, allowing one to correlate differences in gene expression profiles with particular changes in a target gene.

### Utility

As may be apparent, the present method may provide a platform for drug screening, e.g., to identify drugs that make gene expression more wild type. In these embodiments, the method may comprise contacting the genetically modified cells with a drug candidate to determine whether the candidate reverses any changes in gene expression that are caused by the edited base.

The method described herein can be employed to cells from virtually any organism and/or sample-type, including, but not limited to, plants, animals (e.g., reptiles, mammals, insects, worms, fish, etc.). In certain embodiments, the cells used in the method may be derived from a mammal, where in certain embodiments the mammal is a human. In exemplary embodiments, the sample may contain mammalian cells, such as, a human, mouse, rat, or monkey cell. The sample may be made from cultured cells or blood cells.

In some embodiments, the method may be used to analyze different samples, wherein the different samples may include an “experimental” sample, i.e., a sample of interest, and a “control” sample to which the experimental sample may be compared. Exemplary cell type pairs include, for example, cells that have been treated (e.g., with a test agents such as a peptide, small molecule, antibody, hormone, altered temperature, growth condition, physical stress, cellular transformation, etc.), and a normal cell (e.g., a cell that is otherwise identical to the experimental cell except that it is treated, etc.).

Candidate agents that may be used in the method include, but are not limited to, small organic or inorganic compounds having a molecular weight of more than 50 and less than about 2,500 Da. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents may obtained from a wide variety of sources including libraries of synthetic or natural compounds. For example, numerous means are available for random and directed synthesis of a wide variety of organic compounds and biomolecules, including expression of randomized oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling.

In some embodiments, the candidate agent used in the assay may include:

Exemplary agents that can be employed in this method include:
(i) antiproliferative/antineoplastic drugs such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5□-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethox-y]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-met-hylpyrimidin-4-ylamino}thiazole-5-carboxamide (dasatinib, BMS-354825; J. Med. Chem., 2004, 47, 6658-6661), and bosutinib (SKI-606), and metalloproteinase inhibitors like marimastat, inhibitors of urokinase plasminogen activator receptor function or antibodies to Heparanase);
(iv) inhibitors of growth factor function: for example, such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4- -amine (gefitinib, ZD1839), N-(3-ethynylphenyl)-6,7-bis(2-methoxyethoxy)quinazolin-4-amine (erlotinib, OSI-774), and 6-acrylamido-N-(3-chloro-4-fluorophenyl)-7-(3-morpholinopropoxy)-quinazol-in-4-amine (CI 1033), and erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, ab1 kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, for example the anti-vascular endothelial cell growth factor antibody bevacizumab (Avastin) and for example a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)-quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

The bioactive agent used in the method may be an antitumor alkylating agent, antitumor antimetabolite, antitumor antibiotic, plant-derived antitumor agent, antitumor platinum complex, antitumor camptothecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof. In some embodiments, the anti-tumor agent is citabine, capecitabine, valopicitabine or gemcitabine. In some embodiments, the agent is selected from the group consisting of Avastin, Sutent, Nexavar, Recentin, ABT-869, Axitinib, Irinotecan, topotecan, paclitaxel, docetaxel, lapatinib, Herceptin, lapatinib, tamoxifen, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

In one embodiment, the one cell may be used to establish a gene expression profile for a particular mutation, and the effect of the test compounds may be measured, particularly as to whether the compounds provide the cell with a more “wild-type” appearance and may resemble controls that are not contacted with the agent. For example, if a mutation increases the expression of genes involved in the cell cycle or genes downstream thereof, then an agent that reverses that phenotype may be valuable.

Agents that modulate a phenotype may decrease the phenotype by at least 10%, at least 20%, at least 30%, at least 40%, at least 50%, at least 60%, at least 70%, at least 80%, or at least 90%, or more, relative to a control that has not been exposed to the agent.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

Cell culture conditions: HEK293T (ATCC CRL-11268) and MMNK-1 (JCRB1554) cells were maintained in Dulbecco's modified Eagle's medium (DMEM) with 10% fetal bovine serum (FBS). HCT116 (ATCC CCL-247) cells and U2OS (ATCC HTB-96) were maintained in McCoy's 5A modified medium supplemented with 10% FBS. The p53 pathway of cells was stimulated with 10 μM of Nutlin-3a. K562 (ATCC CCL-243) cells were maintained in RPMI 1640 with 10% FBS. Cells were authenticated by STR profiling. All cell lines were confirmed by PCR to be free of mycoplasma contamination.

Lentiviral gRNA library production: The oligonucleotides for sgRNA library generation were ordered using IDT oPools Oligo Pools (Coralville, Iowa, USA). Amplified gRNA cassettes were cloned using NEBuilder HiFi DNA Assembly Master Mix (New England Biolabs, Ipswich, MA, USA) into lentiGuide-Puro (Addgene plasmid #52963). Purified plasmids were electroporated to ElectroMAX Stbl4 Competent Cells (New England Biolabs) and amplified.

Lentivirus production: Approximately 2.0×10⁶ HEK293T cells were plated 24 h prior to transfection. Cells were transfected with pMD2.G (500 ng, Addgene plasmid #12259), psPAX2 (1500 ng, Addgene plasmid #12260) and lentiviral sgRNA library (2000 ng) using Lipofectamine 2000 (Invitrogen) as per the manufacturer's protocol. The viral supernatant was collected after 48 hr of transfection. The supernatants were filtered through a 0.45 μm filter and transduced to cells.

Lentivirus transduction: HCT116 and U2OS cells were diluted to 1.4×10⁵ and 0.7×10⁵ cells/mL and plated a day prior to the transduction. Lentiviral supernatant and polybrene (8 μg/mL, Sigma-Aldrich, MO, USA) were added to the cells. After 24 hours, transduced cells were selected by puromycin (Life technologies, CA, USA) at concentration of 0.4 μg/mL and 1.0 μg/mL.

Transfection and electroporation condition: 1.2×10⁶ HEK293T cells were used to transfect the base editor plasmids (2000 ng) using Lipofectamine 2000 (Invitrogen, Carlsbad, CA, USA) as per the manufacturer's protocol. 1.0×10⁶ HCT116, U2OS and K562 cells were used to transfect the base editor plasmids (2600 ng) using SE or SF solution and 4D-nucleofector (Lonza, Switzerland) as per the manufacturer's protocol. SE solution and DN-100 program were used for MMNK-1 cells. Base editor plasmids pCMV_AncBE4max_P2A_GFP and pCMV_ABEmax_P2A_GFP were gifts from David Liu (Addgene plasmid #112100 and 112101).³⁴ Base editor constructs pCAG-CBE4max-SpG-P2A-EGFP (RTW4552) and pCMV-T7-ABEmax(7.10)-SpG-P2A-EGFP (RTW4562) were gifts from Benjamin Kleinstiver (Addgene plasmid #139998 and #140002). After six days of electroporation, cells were subjected to chemical treatment or single-cell library preparation. For TP53 variant clone generation, base editor plasmids (2250 ng) and sgRNA plasmid (750 ng) were electroporated to cells. Single cell subcloning with limiting dilution was conducted and the genotype of the target was confirmed with PCR amplification and sequencing.

Single-cell library preparation: Single-cell cDNA and gene expression libraries are generated using Chromium Next GEM Single Cell 5′ Library & Gel Bead Kit v2 (10× Genomics, Pleasanton, CA, USA) according to the manufacturer's protocol. The cDNA and gene expression libraries are amplified with 16 and 14 cycles of PCR respectively. The quality of gene expression libraries is confirmed using 2% E-Gel (ThermoFisher Scientific, Waltham, MA, USA). The sequencing libraries were quantified using Qubit (Invitrogen) and sequenced on Illumina sequencers (Illumina, San Diego, CA, USA).

Single-cell sgRNA capture and sequencing: The sgRNA direct capture was performed as previously described. Briefly, six pmol of sgRNA scaffold binding primer was added to RT master mix. After cDNA amplification, the sgRNA fractions were purified using SPRIselect bead (Beckman Coulter Life Sciences, CA, USA). The library was amplified and sequenced with gene expression library.

Long-read sequencing: Ten ng of the single-cell full length cDNA were used to amplify transcripts. Primer sequences are shown in Supplementary Table 5. KAPA HiFi HotStart ReadyMix (Roche, Basel, Switzerland) was used for amplification. Libraries were prepared with 900 fmol of each amplicon for Promethion flow cell FLO-PR0002 (Oxford Nanopore Technologies, Oxford, UK) using Native Barcoding Expansion and Ligation Sequencing Kit (Oxford Nanopore Technologies) according to the manufacturer's protocol. Libraries were sequenced on a Promethion over 72 h.

### Single Cell Transcript Analysis

Short read transcripts: Basecalling for 5′ gene expression libraries was performed using cellranger 6.0 (10× Genomics). In preparation for integrated analysis, the transcript count matrices generated by cellranger were processed by Seurat 3.0.2. QC filtering removed cells with fewer than 100 or more than 8000 genes, cells with more than 30% mitochondrial genes and cells predicted to be doublets by DoubletFinder. Additionally, any genes present in three or fewer cells were removed. Batch effects between each single-cell cDNA generation reaction and base editors were corrected by Harmony. Cell cycle phase were also corrected by Harmony.

Long read variant calling: Basecalling was performed using guppy 5 with super accuracy mode and alignment to the GRCh38 reference genome using minimap2. Cell barcodes and UMIs are extracted as previously described. For validating TP53 mutation genotyping, UMIs with less than 10 reads were filtered out and UMIs with high similarity (edit distance less than 3) were consolidated. A custom python script utilizing the pysam module was used to identify reads spanning the sgRNA target windows and extracted the base calls at each position within the window. Base calls were used predict amino acid changes per each cell. Cells with heterozygous amino acid changes were excluded for the gene expression analysis. Output from this script was summarized to provide expected amino acid change per cell barcode.

Integration of long and short reads: The variant per cell barcode table were added to the Seurat object metadata as a new column. Cells without high-quality long-read data were filtered. For gene expression analysis, variants which were detected in less than 5 cells were filtered. A hierarchical clustering was done in R using hclust, cutree and dendextend. Biological pathway analysis was performed with the Gene Set Variation Analysis (GSVA) tool.

Cell cycle analysis: Click-iT™ Plus EdU Alexa Fluor™ 488 Flow Cytometry Assay Kit (Life technologies) was used according to manufacturer's protocol. Briefly, cells were plated a day prior to nutlin-3a or vehicle treatment. After 24 hrs of chemical treatment, cells in S-phase were labeled with 10 mM EdU solution for 2 hrs. FxCycle™ PI/RNase Staining Solution (Life technologies) was used for PI staining. After the staining, cells were analyzed by NovoCyte Quanteon Flow Cytometer Systems (Agilent, Santa Clara, CA, USA).

RNA sequencing: KAPA mRNA HyperPrep Kit (Roche) was used for mRNA sequencing library preparation according to manufacturer's protocol. For each cell type, triplicate library preparations with 1 g of total RNA were used as an input. Libraries were sequenced by NextSeq (Illumina) by 75 bp paired-end sequencing. The reads were aligned to the reference genome GRCh38 by a two-pass method with STAR and gene expression level was measured using HT-Seq. DESeq2 was used for DE analysis. Biological pathway analysis was performed with the Gene Set Variation Analysis (GSVA) tool.

### Results

Identifying Mutations with Single-Cell cDNA Sequencing

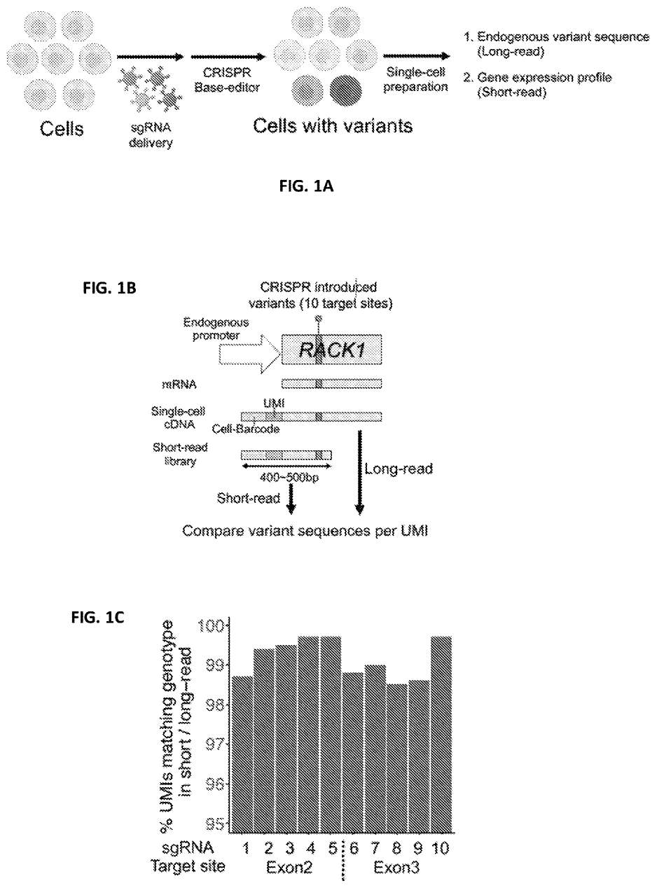

Some principles of the TISCC-Seq method are illustrated in FIG. 1a. An analysis comparing long versus short read single-cell cDNA sequencing was conducted. For this initial test, an assay was designed to introduce different genetic variants in exon2 and 3 of the RACK1 gene (FIG. 1b). The length of RACK1 cDNA up to exon3 is approximately 500 bp—this length interval can be fully covered with short reads. This gene is one of the most highly expressed in the HEK293T cell line as determined from single-cell short- and long-read gene expression data from a previous publication. 10 sgRNAs targeting exon2 and 3 of RACK1 gene were designed and lentiviruses encoding those sgRNAs were transduced to HEK293T cells at 0.1 multiplicity of infection. Transduced cells were selected by puromycin. Then, a plasmid encoding an adenine base editor (ABE) was transfected into the cells. This step introduced multiple genetic variants at sgRNA target sites. After six days, single cell cDNAs were generated and genomic DNA was extracted from cells derived from the same suspension.

From the genomic DNA of transduced cells, exon2 or 3 of the RACK1 gene was amplified and short-read sequencing was performed to evaluate the frequency of genetic variants in RACK1 genomic DNA. Based on the DNA sequencing, genetic variants introduced by all ten sgRNAs were identified. The frequency of ABE-induced genetic variants varied from 1.1% to 10.1% from the genomic DNA of pooled cells (data not shown).

Next, the presence of these variants was evaluated at a single-cell transcript level using single cell cDNAs. These engineered variants were proximal to the 5′ end of the cDNA, allowing sequencing of the variants with short reads (i.e., Illumina). Short read sequences have a high base quality for variant calling and allowed comparison of the long and short read results. From the single-cell cDNA library, sequencing libraries for both short- and long-read sequencing were prepared to assess single-cell level genetic variants from the RACK1 transcripts. For short-read sequencing, exon2 or 3 of RACK1 was amplified from single cell cDNA with cell barcodes and unique molecular index (UMI) sequences using the 5′ adaptor primer and exon specific primers (FIG. 1b). These libraries were sequenced on the Illumina Miseq platform. In Illumina sequencing, each DNA fragment is sequenced from both ends, resulting in two reads per fragment. These two reads are referred to as read 1 and read 2. Similar to regular single-cell gene expression sequencing, 26 bp of read1 sequences were used for cell barcode and UMI extraction. The read2 sequences were used for the evaluation of the newly introduced RACK1 genetic variants at target sites. Using the genetic coordinates of the sgRNA target window (i.e., 3 bp to 8 bp), for a given read, the corresponding cell barcode, UMI and the genetic variant were identified.

For long-read sequencing, the entire RACK1 cDNA was amplified using the 5′ adaptor and primers specific to the last 3′ exon from the same single cell cDNA library (FIG. 1b). The intact cDNA amplicon was sequenced with an Oxford Nanopore instrument. Guppy was used for base calling and minimap2 was used for alignment. Each sequence read had the cell barcode, UMI and complete RACK1 cDNA sequence. The cell barcodes and UMI were extracted as previously described.⁷ After genome alignment of the long-read data, the cell barcodes and UMI fell into soft-clipped sequence. Therefore, the soft-clipped portion of each read was extracted and compared with the cell barcodes identified from gene expression library sequencing. Only reads with perfectly matching cell barcodes were used for further analysis. Using the aligned long-read data, the RACK1 genetic variants were identified. Therefore, long read information provided the genetic variants with accompanying cell barcode and UMI sequence. For additional quality control filtering, UMIs with less than three reads were filtered out. Consensus genetic variants for each UMI were generated using multiple reads.

The RACK1 variant calls from short- and long-read single cell data were compared. Consensus RACK1 genetic variants were analyzed for each cell barcode and UMI combination. Across all target sites, 479,509 UMIs were compared: 99.2% of them had identical genetic variants in average (FIG. 1c). This result demonstrated the high accuracy of long read identification of CRISPR-engineered genetic variants. Recent improvements in the accuracy of nanopore sequencing and UMI based consensus generation enabled this analysis. The frequency of genetic variants from genomic DNA and aggregated single-cell cDNA were then compared for each of the 10 target sites introduced by base editors. The frequency of each variant between genomic DNA and single-cell cDNA had a high correlation (R²=0.63).

### Base Editor Guide RNA Designs for TP53 Cancer Mutations

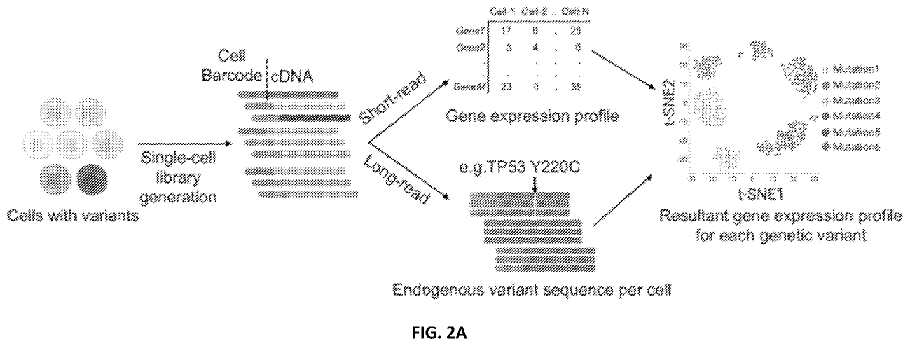

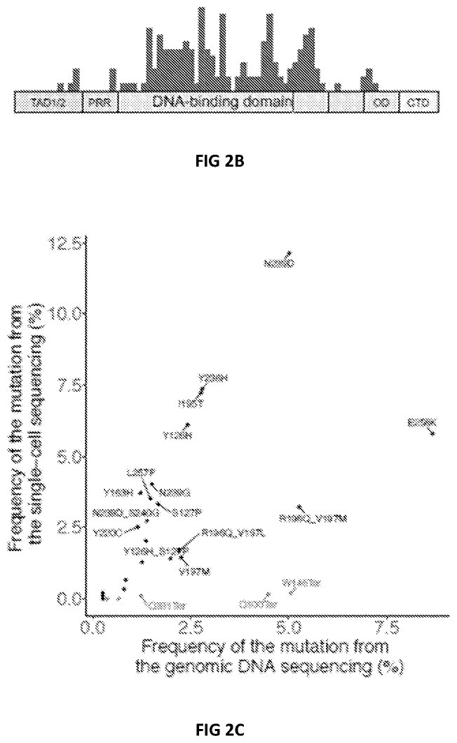

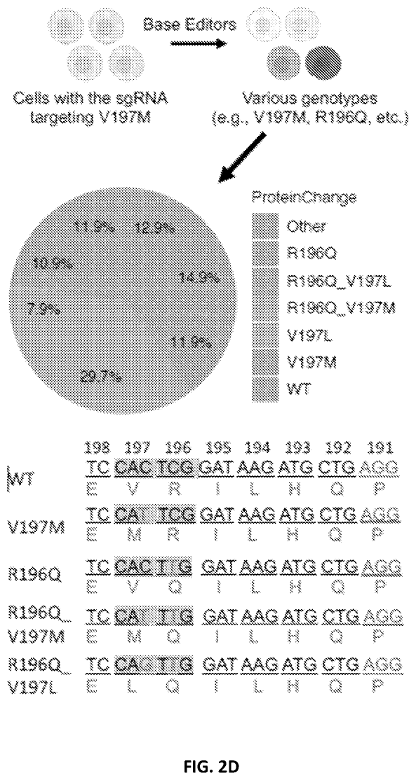

A set of sgRNAs designed for multiple TP53 mutations were introduced and TISCC-Seq was used to obtain the gene expression profile and TP53 genotype from individual cells. First, the design of the genome engineering of TP53 mutations was focused on (FIG. 2a). TP53 mutations which were reported more than nine times in the COSMIC database were identified. The majority of these frequent cancer mutations were within the TP53 DNA-binding domain. The total number of coding mutations was 351. Base editor libraries targeting this mutation set were designed. To cover as many mutations as possible, several base editor combinations were used: (1) CBE with NGG protospacer adjacent motif (PAM); (2) CBE with a NG PAM; (3) ABE with NGG PAM; (4) ABE with a NG PAM. Using the NGG PAM base editors, 74 sgRNAs targeting 99 TP53 variants were designed. The NG PAM base editors have more flexible PAM, enabling design of an additional 88 sgRNAs targeting 159 variants (data not shown). Most of sgRNAs targeted the DNA binding domain of p53 protein (FIG. 2b).

Base editors can alter any target nucleotide in their target window (i.e., 3 bp to 8 bp) which leads to different nucleotides at that position. TISCC-seq identified this variation among single cells. For example, the sgRNA introducing E258K mutation by C to T substitution induces the E258G mutation by C to G substitution (data not shown). Similarly, the sgRNA introducing S127P mutation by A to G substitution at the 3^{rd} adenine induces the Y126H mutation by A to G substitution at the 6^{th} adenine (data not shown). Therefore, this result suggests that any given sgRNA can introduce multiple variants depending on the window sequence context. The entire number of amino acid changes that could be introduced by the NGG or NG PAM base editors and the sgRNA libraries were 920 and 1999 respectively. For the final design, 251 known TP53 mutations were targeted with the potential for introducing 2892 possible amino acid changes (data not shown).

### CRISPR Base Editor Engineering of TP53 Mutations

HCT116 and U2OS human cell lines were used for this study. Both cell lines have wild-type TP53 which was independently confirmed. The p53 pathway is repressed by the negative regulator MDM2 in both cell lines. The oncoprotein MDM2 is an E2 ubiquitin ligase.¹⁵ It binds to and promotes the ubiquitin-dependent degradation of the p53 protein. The small molecule nutlin-3a can inhibit p53-MDM2 binding efficiently. To activate the p53 pathway and select for TP53 mutations with functional effects, various concentrations of nutlin-3a were tested, including 5 μM, 10 μM, and 20 μM, based on previous reports. The results showed successful p53 pathway activation at 10 μM nutlin-3a, which was used for both cell lines.

Four sgRNA libraries were generated for each base editor (NGG-CBE, NGG-ABE, NG-CBE, NG-ABE)—the combined libraries were designed to cover the preselected TP53 mutations. Those libraries were transduced using a lentivirus system to both the HCT116 and U2OS cell lines. The cells were transfected with each respective base editor plasmids. It had been reported that base editors can induce off-target RNA editing. To minimize those effects, transient transfection was chosen rather than stable expression of base editors. Typically, plasmid based protein expression peaks after 24 hrs of transfection and diminishes after 5-6 days. Six days after transfection, nutlin-3a was used to activate the p53 pathway.

### TISCC-Seq Detection of TP53 Mutations

After 10 days of nutlin-3a treatment, the cells were harvested for suspension, single-cell cDNA libraries were prepared and genomic DNA was also extracted from a portion of the cell suspension. TP53 transcripts were amplified from the single-cell cDNA library, their full-length transcript was sequenced and the presence of the TP53 mutation was determined from the long read data (FIG. 2a). As an important additional step, cell barcodes and UMI per each long-read were extracted as described earlier. To prevent the effect of sequencing error in UMI region, any UMI with less than 10 long reads was filtered out. As a quality control threshold, only the cell barcode and UMI combinations found in 10 or more reads were used. For generating a consensus, UMIs with a low edit distance were also included, assuming the differences were related to sequencing errors. For TP53 variant calling, every nucleotide sequence in the sgRNA target window (e.g., chr 17:7674940-7674945 for the sgRNA in FIG. 2d) was extracted and compared with the reference sequence (e.g., CACTCG to CATTCG). Based on nucleotide changes of a given mutation, the amino acid substitution at the target site (e.g., V196M) was determined.

For independent validation, amplicon sequencing from the transduced cells' genomic DNA was used to independently assess the frequency of a subset of TP53 mutations. This analysis compared the frequency of each TP53 mutations introduced by 12 sgRNAs in genomic DNA versus the results from analyzing the single-cell cDNA from HCT116 cells. These TP53 mutations were introduced efficiently with up to 12.1% for one variant and 27 variants were introduced with a frequency greater than 0.25%. The prevalence of each mutation from single-cell cDNA and genomic DNA was generally correlated (FIG. 2c, R²=0.59). Some variants had higher frequency in genomic DNA and lower in cDNA (i.e., W146Ter). This result means that for some mutations the corresponding transcripts were not expressed efficiently or were subjected to higher RNA degradation. The lower prevalence of cDNA mutations may reflect effects from nonsense mediated decay (NMD). This process is a surveillance mechanism that eliminates mRNA transcripts containing premature stop codons. For example, although 5.1% of cells had a W146Ter mutation at the genomic DNA level, this mutation was not detected as frequently at the single cDNA level (0.2%) because the transcripts with the variant were degraded in cells by NMD (FIG. 2c).

As another type of validation, the sgRNA expressed in each cell was sequenced from single-cell cDNA using a direct capture method previously described. Most of the single-cell CRISPR screen studies have relied on an sgRNA sequencing method to infer the resultant genetic edits. This method assumes that cells with the sgRNA have the targeted genomic edit. However, the efficiency of base editors is lower than Cas9 nuclease. As described earlier, a base editor may introduce multiple genetic variants from the same sgRNA (data not shown). Therefore, one cannot assume that cells transduced with base editors and a single sgRNA have the intended variant at the target position (FIG. 2d). The results showed that this was the case. For example, a sgRNA which was designed to introduce the TP53 V197M mutation was evaluated. The sgRNA's target site has three cytosines in its window. Among 101 cells expressing this specific sgRNA, 11 cells had V197M mutation while 30 cells had both R196Q and V197M mutations (FIG. 2d). Therefore, the conventional single-cell CRISPR screening method using sgRNA sequencing did not correctly identify the introduced variants among the various single cells. In contrast, with direct long read sequencing of the full-length target transcripts from single cells, this issue is bypassed and the actual mutation introduced by the base editor is directly identified from the cDNA.

TISCC-Seq and Analysis of HCT116 Cells with TP53 Mutations

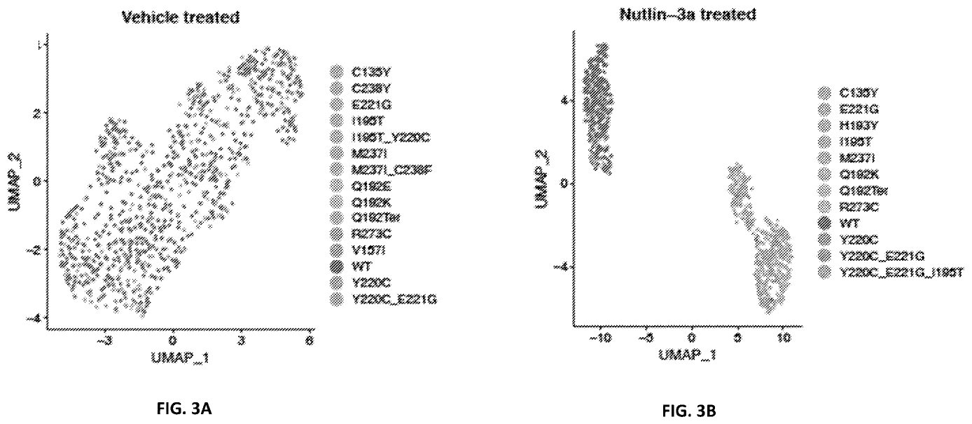

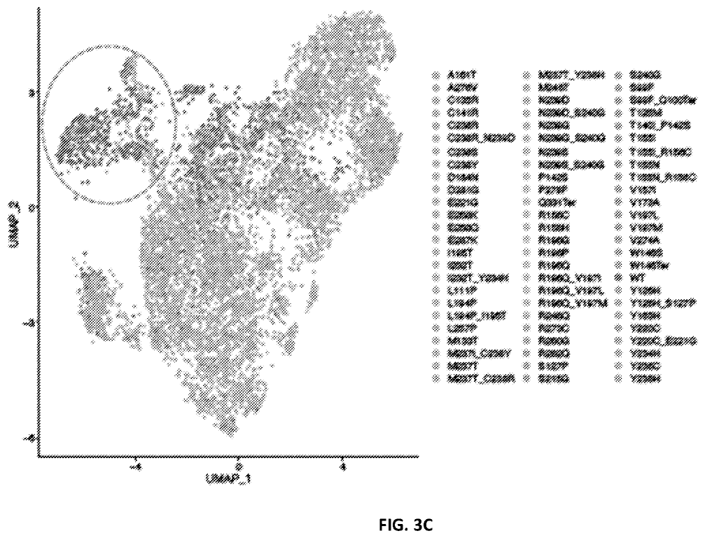

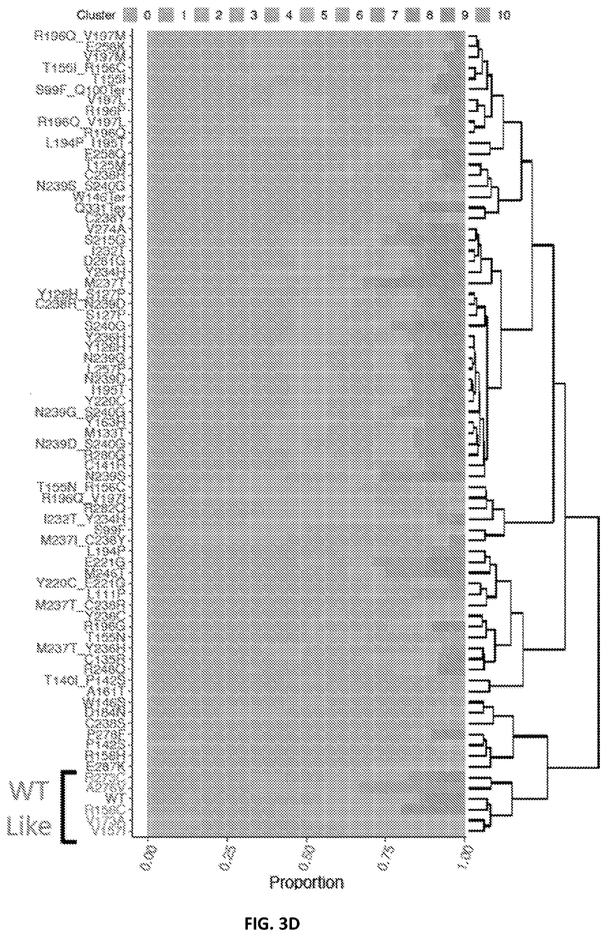

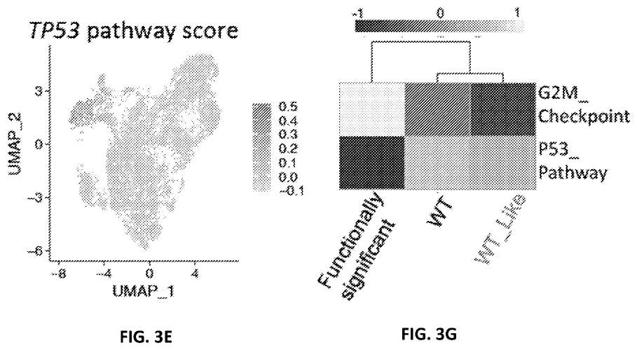

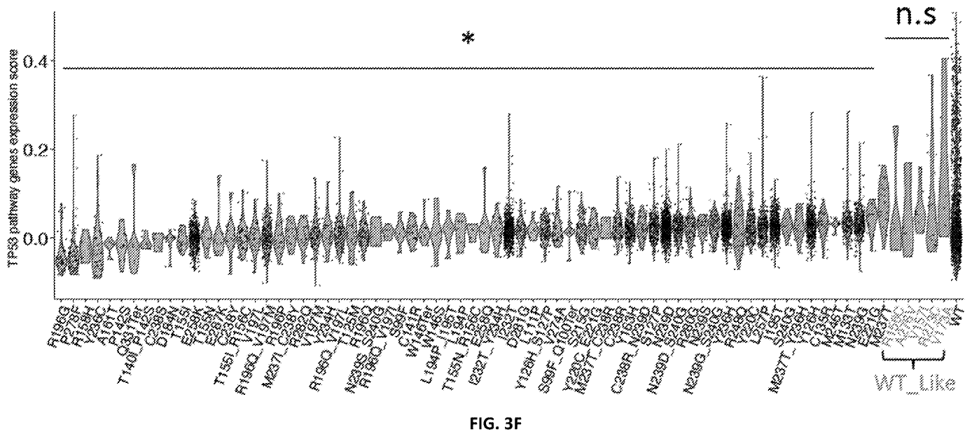

Gene expression analysis was performed using the same single-cell cDNA library used for long-read sequencing. As described previously, the single cell TP53 mutation genotypes from long reads were integrated with the single-cell gene expression profile data from short reads. Cell barcode matching between the long read data with a mutation genotype and the short read data was used. This process allows linking those cells with TP53 mutation to their individual gene expression profiles. To conduct a cluster analysis of the cells with different TP53 mutations, Uniform Manifold Approximation and Projection (UMAP) was used (FIG. 3). The effect of p53 pathway activation by nutlin-3a in HCT116 cells with TP53 mutations was investigated using a subset of our sgRNA library (10 sgRNAs). When the gene expression profiles between cells with wild-type or TP53 mutations was compared, there was a significant and clearly delineated difference upon p53 pathway activation (FIGS. 3a and 3b). When the expression of p53 pathway involved genes was visualized on a UMAP plot using a heatmap, it was found that cells with deleterious TP53 mutations displayed decreased p53 pathway involved gene expression compared to wild-type cells (data not shown).

Next, HCT116 cells transduced with the full TP53 sgRNA library and activated by nutlin-3a were sequenced. Among the 42,564 cells that were sequenced, a set of high quality long read UMIs (UMI read count>9) covering TP53 from 12,887 cells were filtered out. This subset of high quality reads were useful for confirming the mutation genotype. Each cell had an average of 898 TP53 reads with a complexity of 4.5 UMIs for this subset. Cells which had a heterozygous mutation were filtered out. Overall, a total of 169 different mutations distributed among the various single cells were detected.

Single cell gene expression for each mutation was analyzed. To provide a robust measurement of single cell expression, those TP53 mutations expressed in fewer than five cells were filtered out. This step retained 74 mutations for further analysis. Via UMAP clustering, the cells with wild-type versus TP53 mutations separated among different clusters. Compared to the clustering observed in FIG. 3b, which included 11 mutations, this dataset encompasses 74 mutations with a wider range of impact. As a result, the separation between wild-type cells and other cells is less distinct in this dataset. Wild-type cells were predominantly clustered in Cluster 5 and 9 (FIG. 3c). For each variant, its proportion within each cluster was calculated and hierarchical clustering of each variant was performed based on the proportion (FIG. 3d). Cells with the following five mutations (R156C, V157I, V173A, R273C and A276V) clustered with the wild type cells. This result was a preliminary indication that this set of mutations did not have a significant impact on the gene expression phenotype—they were annotated as wild-type like and the others as functionally significant.

The expression of 343 genes known to be involved in the p53 pathway from a previous report using single cell data analysis (data not shown) were examined. Cells that were wild type or with mutations that were wild type like had higher expression of p53 pathway involved genes (FIG. 3e). Wild-type cells had higher p53 pathway gene expressions score compared to the majority of cells expressing functionally significant TP53 mutations (FIG. 3f, P<0.03). Additionally, the expression of the CDKN1A gene, which encodes a p21 protein, was analyzed. p21 protein is a regulator of cell cycle progression and arrest. Wild-type cells had higher CDKN1A expression compared to the cells with functionally significant TP53 mutations. Next, pathway analysis was performed between wild-type cell and cells with wild-type like versus functionally significant variants. Cells with functionally significant mutations had lower p53 pathway activity and higher G2M checkpoint gene expression than the wild-type cells (FIG. 3g, P=1.66e-11 and 1.66e-11). In addition, cells with wild-type like variants expressing the R156C, V157I, V173A, R273C or A276V did not have differences on two pathways compared to cells with wild type TP53 (FIG. 3g, P=0.95 and 0.44). These results are evidence that this subset of the mutations had features similar to wild type and thus had less functional impact. In summary, wild-type cells had higher active p53 pathway activity and related gene expression than cells with functionally significant TP53 variants. These results validated the TISCC-seq method for high throughput functional classification of these mutations.

### TISCC-Seq Analysis of TP53 Mutations in U2OS Cell Line

As an additional verification of the results, a similar analysis was performed with the U2OS cell line using the same sgRNAs for the TP53 mutations. Among 38,451 cells that were sequenced, high quality long-read sequences from 12,155 cells were acquired. On average per each cell, the high quality TP53 reads, of which there were 890 with a complexity of 4.6 UMIs, were filtered out. As described, a filtering strategy was applied to eliminated heterozygous mutations. For the U2OS line, 161 mutations were characterized with TISCC-seq. For gene expression analysis, the 62 variants which were detected in more than five cells were used. From the UMAP analysis, wild-type cells and cells with TP53 mutations separate into distinct clusters (data not shown). Wild-type cells were primarily associated with Cluster 1. For each mutation, its proportion within each cluster was calculated and hierarchical clustering was performed based on this cluster proportion (data not shown). From the hierarchical clustering results, four mutations were identified, T140I, R156C, T221I and R273C, that were associated with wild type TP53. The R156C and R273C mutations had a similar association with the wild type cells for both the HCT116 and U2OS cell lines. The wild-type U2OS cells had higher expression of CDKN1A and other p53 pathway involved genes compared to the majority of cells expressing functionally significant TP53 mutations (data not shown). The analysis of pathway activity showed that cells with functionally significant mutations had significantly lower p53 pathway activity and higher G2M checkpoint gene expression (P=1.62e-12 and 1.62e-12). Conversely, cells with wild-type like mutations were not statistically significant to the same extreme degree as the functionally significant mutations (P=0.52 and 0.001).

### Confirmation of TISCC-Seq Using Clonal Cell Lines

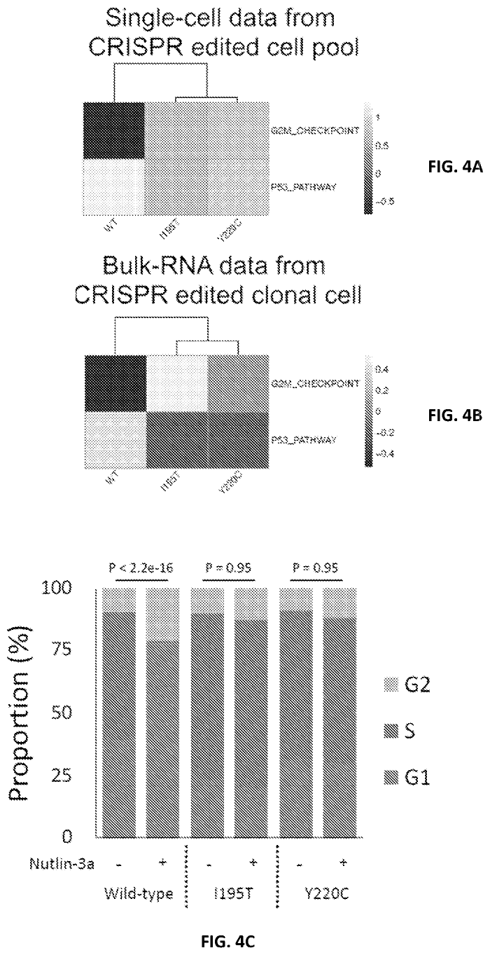

The prior experiments were highly multiplexed in engineering different mutations. Providing additional confirmation of the single cell results, simplex experiments of individual mutations were conducted using the HCT116 cell line. Using the ABE, homozygous clonal cell lines were generated with either the TP53 I195T or Y220C mutation which were functionally significant and had enough cells from single-cell assay. To obtain clones, limiting dilution after ABE transfection was used. These two mutations have been reported to have a deleterious effect on function and the multiplexed TISCC-seq results also demonstrated that they had a functional effect (FIG. 3d). Bulk-RNA seq was performed from nutlin-3a treated wild-type cells and those clonal cells. The result with single cells was compared with results from HCT116 cell-lines (FIG. 4).

From the single cell results, both mutations demonstrated lower p53 pathway activity and higher G2M checkpoint gene expression than wild-type cells (FIG. 4a, I195T: P=2.2e-11 and 1.7e-3. Y220C: 2.2e-11 and 9.4c-2). From the conventional, bulk-based RNA-seq results, the same effect on the same pathways was observed (FIG. 4b, I195T: P=3.4e-6 and 2.4e-7. Y220C: 1.0e-4 and 2.6e-7). Next, differential gene expression (DGE) analysis was performed between wild-type versus mutation-bearing cells. The DGE results from scRNA-seq and standard RNA-seq was compared. For the I195T or the Y220C mutations, the top 100 genes determined from single cell RNA-seq data were identified. For the I195T mutation, 94 out of 100 were confirmed as showing differential expression per the conventional RNA-seq. Likewise for the Y220C mutation, 80 out of 100 genes were confirmed as showing differentially expression per the conventional RNA-seq (P<1.0e-5).

Overall, the I195T and Y220C cell lines had higher G2M checkpoint gene expression as an indicator of more active cell division compared to the cells with wild type TP53. To validate this result, cell division and cell cycling from wild-type and TP53-mutated HCT116 cells was evaluated using 5-ethynyl-2′-deoxyuridine (Edu) and a propidium iodide (PI) flow cytometry assay. The PI assay detects total DNA amounts for G1 and G2-phase comparison. The EdU assay labels newly synthesized DNA to detect S-phase. The cell cycle of wild-type HCT1 16 cells was arrested by nutlin-3a treatment (FIG. 4c, P<2.2e-16). In contrast, the cell cycle of HCT116 cells with either the I195T or the Y220C mutations did not undergo arrest with nutlin-3a treatment (FIG. 4C, P=0.95 and 0.95).

The analysis was expanded by generating five additional clones with TP53 mutations and RNA sequencing analysis was conducted (data not shown). The V157I mutation was categorized as wild-type like, while the remaining mutations were deemed functionally significant based on the TISCC-seq analysis. The results revealed that HCT116 cells with the V157I mutation exhibited a gene expression profile that was similar to wild-type cells, while cells with functionally significant mutations showed distinct differences in gene expression. To further investigate the impact of TP53 mutations on cell growth, growth assays were conducted using HCT116 cells with ten different TP53 mutations which were categorized as functionally significant (data not shown). The data demonstrated that cells with these mutations exhibited a growth advantage over wild-type cells when treated with nutlin-3a, further supporting the notion that these mutations confer a growth advantage. This result established that this single cell approach accurately identified the phenotypes of these mutations.

### DISCUSSION

In this study, a multiplexed method that uses base editors to introduce specific cancer mutations and single-cell sequencing to identify the genotype and phenotypes of the induced cancer mutations is demonstrated. Referred to as TISCC-seq, this approach overcomes issues with short-read based single-cell or bulk CRISPR screens, neither of which verify endogenous DNA variants that are engineered into the genomes of cells. This approach integrated single-cell long-read and short-read sequencing for CRISPR base editor screens. As a result, endogenous genetic variants introduced by the CRISPR base editor are directly confirmed from the target gene transcript. At single-cell resolution, the genetic variant and its resultant transcriptome changes become evident. Therefore, the functional consequences of TP53 mutations can be determined across different cell lines. Some mutations had a greater functional impact on the cells' gene expression while a smaller subset had a wild-type like phenotype. The results corroborated some in silico predictions (data not shown). For example, the R156C mutation is predicted to have neutral effect on p53 pathway. This was confirmed experimentally among the results. In both cell lines used in this study, this mutation had a wild-type phenotype. Overall, this approach has the potential for enabling highly multiplexed functional evaluation of cancer mutations and germline variants. Following functional assays using cell lines with desired genetic variants will help deeper understanding of the phenotype of each variant as shown in FIG. 4.

Although four base editors were used for this study, there were some mutations that were unable to be targeted (data not shown). It is anticipated that modification of base editor properties such as their enzymatic activity, window and PAM restriction will broaden the types of mutations and other variants which can be engineered into genomes. The prime editor which can introduce any genetic variant at the target site will even enable saturation mutagenesis of the target gene.

Mutually exclusive TP53 mutations were observed in HCT116 and U2OS cell lines through TISCC-seq analysis (data not shown). The analysis suggests that differences in CRISPR base editing efficiencies between the two cell lines may account for these mutations. For instance, the C135Y mutation, which was only detected in U2OS cells and deemed functionally significant, exhibited low editing efficiency (~1%) when attempted to introduce it into HCT116 cells using a guide RNA with a CRISPR base editor. Consequently, the mutation was not observed in the HCT116 cell TISCC-seq data. Nevertheless, the findings revealed that the C135Y mutation conferred a growth advantage in HCT116 cells. Four functionally significant TP53 mutations (I195T, Y220C, Y236H, and L257P) were investigated in non-cancer MMNK1 cells. These cells were treated with nutlin-3a and no evidence of a growth advantage was found in cells carrying these TP53 mutations. This observation is consistent with the known role of the p53 pathway, which frequently triggers cell-cycle arrest or apoptosis in response to various stresses that are more prevalent in developed cancer cells than in non-cancer cells. The results underscore the potential utility of TISCC-seq in revealing the functional consequences of mutations across diverse cellular contexts, including primary cells and developed cancer cells.

It was further demonstrated that TISCC-seq can be applied to longer genes by targeting SF3B1, which has a transcript longer than 6 kb, and introducing multiple mutations using CRISPR base editors in K562 cells. The analysis using TISCC-seq successfully genotyped these mutations at the single-cell level. These results illustrate the versatility of TISCC-seq and its potential to enable the assessment of genetic variants across a broad range of genomic contexts, including longer genes.

The complexities of high-throughput CRISPR engineering, single-cell sequencing and its higher cost limit the scalability of single-cell CRISPR screens compared to conventional genetic screens done with conventional bulk assays. TISCC-seq provides some potential benefits that may be useful for standard CRISPR screens. For example, one can use a bulk-based cellular genetic screen for hundreds of thousands sgRNAs generating variants and then narrow down the sgRNAs to the hundreds with significant impact on cell survival or drug response. Then, TISCC-seq can be used for a deeper analysis of sgRNAs by detecting genuine endogenous mutations and their resultant phenotype at single-cell level resolution. This combination may enable more accurate evaluation of CRISPR-based screens in the future.

The sensitivity of single-cell RNA sequencing is limited. Therefore, only a limited number of transcripts for each gene can be detected. It is challenging to detect any transcripts from low-expressed genes in individual cells. This sparsity in single-cell RNA sequencing data restricts the application of TISCC-seq to genes with extremely low expression levels. However, advancements in single-cell reverse transcription and transcript enrichment technology can greatly enhance the efficiency of TISCC-seq.

### REFERENCES

1. Cuella-Martin, R. et al. Functional interrogation of DNA damage response variants with base editing screens. Cell 184, 1081-1097 e1019 (2021).
2. Hanna, R. E. et al. Massively parallel assessment of human variants with base editor screens. Cell 184, 1064-1080 e1020 (2021).
3. Kim, Y. et al. High-throughput functional evaluation of human cancer-associated mutations using base editors. Nat Biotechnol 40, 874-884 (2022).
4. Sanchez-Rivera, F. J. et al. Base editing sensor libraries for high-throughput engineering and functional analysis of cancer-associated single nucleotide variants. Nat Biotechnol 40, 862-873 (2022).
5. Ursu, O. et al. Massively parallel phenotyping of coding variants in cancer with Perturb-seq. Nat Biotechnol 40, 896-905 (2022).
6. Hill, A. J. et al. On the design of CRISPR-based single-cell molecular screens. Nat Methods 15, 271-274 (2018).
7. Kim, H. S., Grimes, S. M., Hooker, A. C., Lau, B. T. & Ji, H. P. Single-cell characterization of CRISPR-modified transcript isoforms with nanopore sequencing. Genome Biol 22, 331 (2021).
8. Wick, R. R., Judd, L. M. & Holt, K. E. Performance of neural network basecalling tools for Oxford Nanopore sequencing. Genome Biol 20, 129 (2019).
9. Li, H. Minimap2: pairwise alignment for nucleotide sequences. Bioinformatics 34, 3094-3100 (2018).
10. Tate, J. G. et al. COSMIC: the Catalogue Of Somatic Mutations In Cancer. Nucleic Acids Res 47, D941-D947 (2019).
11. Berglind, H., Pawitan, Y., Kato, S., Ishioka, C. & Soussi, T. Analysis of p53 mutation status in human cancer cell lines: a paradigm for cell line cross-contamination. Cancer Biol Ther 7, 699-708 (2008).
12. de Andrade, K. C. et al. The TP53 Database: transition from the International Agency for Research on Cancer to the US National Cancer Institute. Cell Death Differ 29, 1071-1073 (2022).
13. Leroy, B. et al. Analysis of TP53 mutation status in human cancer cell lines: a reassessment. Hum Mutat 35, 756-765 (2014).
14. Tovar, C. et al. Small-molecule MDM2 antagonists reveal aberrant p53 signaling in cancer: implications for therapy. Proc Natl Acad Sci USA 103, 1888-1893 (2006).
15. Honda, R., Tanaka, H. & Yasuda, H. Oncoprotein MDM2 is a ubiquitin ligase E3 for tumor suppressor p53. FEBS Lett 420, 25-27 (1997).
16. Vassilev, L. T. et al. In vivo activation of the p53 pathway by small-molecule antagonists of MDM2. Science 303, 844-848 (2004).
17. Grunewald, J. et al. Transcriptome-wide off-target RNA editing induced by CRISPR-guided DNA base editors. Nature 569, 433-437 (2019).
18. Kim, S., Kim, D., Cho, S. W., Kim, J. & Kim, J. S. Highly efficient RNA-guided genome editing in human cells via delivery of purified Cas9 ribonucleoproteins. Genome Res 24, 1012-1019 (2014).
19. Replogle, J. M. et al. Combinatorial single-cell CRISPR screens by direct guide RNA capture and targeted sequencing. Nat Biotechnol 38, 954-961 (2020).
20. Adamson, B. et al. A Multiplexed Single-Cell CRISPR Screening Platform Enables Systematic Dissection of the Unfolded Protein Response. Cell 167, 1867-1882 e1821 (2016).
21. Datlinger, P. et al. Pooled CRISPR screening with single-cell transcriptome readout. Nat Methods 14, 297-301 (2017).
22. Jaitin, D. A. et al. Dissecting Immune Circuits by Linking CRISPR-Pooled Screens with Single-Cell RNA-Seq. Cell 167, 1883-1896 e1815 (2016).
23. Rubin. A. J. et al. Coupled Single-Cell CRISPR Screening and Epigenomic Profiling Reveals Causal Gene Regulatory Networks. Cell 176, 361-376 e317 (2019).
24. Kim, H. K. et al. SpCas9 activity prediction by DeepSpCas9, a deep learning-based model with high generalization performance. Sci Adv 5, eaax9249 (2019).
25. Song, M. et al. Sequence-specific prediction of the efficiencies of adenine and cytosine base editors. Nat Biotechnol 38, 1037-1043 (2020).
26. Fischer, M. Census and evaluation of p53 target genes. Oncogene 36, 3943-3956 (2017).
27. Landrum, M. J. et al. ClinVar: public archive of interpretations of clinically relevant variants. Nucleic Acids Res 44, D862-868 (2016).
28. Kakudo, Y., Shibata, H., Otsuka, K., Kato, S. & Ishioka, C. Lack of correlation between p53-dependent transcriptional activity and the ability to induce apoptosis among 179 mutant p53s. Cancer Res 65, 2108-2114 (2005).
29. Richter, M. F. et al. Phage-assisted evolution of an adenine base editor with improved Cas domain compatibility and activity. Nat Biotechnol 38. 883-891 (2020).
30. Thuronyi, B. W. et al. Continuous evolution of base editors with expanded target compatibility and improved activity. Nat Biotechnol 37, 1070-1079 (2019).
31. Huang, T. P. et al. Circularly permuted and PAM-modified Cas9 variants broaden the targeting scope of base editors. Nat Biotechnol 37, 626-631 (2019).
32. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
33. Anzalone, A. V. et al. Search-and-replace genome editing without double-strand breaks or donor DNA. Nature 576, 149-157 (2019).
34. Koblan, L. W. et al. Improving cytidine and adenine base editors by expression optimization and ancestral reconstruction. Nat Biotechnol 36, 843-846 (2018).
35. Stuart, T. et al. Comprehensive Integration of Single-Cell Data. Cell 177, 1888-1902 e1821 (2019).
36. McGinnis, C. S., Murrow, L. M. & Gartner, Z. J. DoubletFinder: Doublet Detection in Single-Cell RNA Sequencing Data Using Artificial Nearest Neighbors. Cell Syst 8, 329-337 e324 (2019).
37. Korsunsky, I. et al. Fast, sensitive and accurate integration of single-cell data with Harmony. Nat Methods 16, 1289-1296 (2019).
38. Hanzelmann, S., Castelo, R. & Guinney, J. GSVA: gene set variation analysis for microarray and RNA-seq data. BMC Bioinformatics 14, 7 (2013).
39. Dobin, A. et al. STAR: ultrafast universal RNA-seq aligner. Bioinformatics 29, 15-21 (2013).
40. Anders, S., Pyl, P. T. & Huber, W. HTSeq—a Python framework to work with high-throughput sequencing data. Bioinformatics 31, 166-169 (2015).
41. Love, M. I., Huber, W. & Anders, S. Moderated estimation of fold change and dispersion for RNA-seq data with DESeq2. Genome Biol 15, 550 (2014).
42. Kim, H. S., Grimes, S. M., Chen, T., Sathe, A., Lau, B. T., Hwang, G.-H., Bae, S., Ji, H. P. Direct measurement of engineered cancer mutations and their transcriptional phenotypes in single cells. Dataset. Sequence Read Archive (SRA).

1. A method for analyzing cells, comprising:
(a) base editing a target gene in a population of cells to produce genetically modified cells;
(b) reverse transcribing mRNA from single cells in the population of cells to produce cDNA, wherein the cDNA produced by each cell has a cell barcode and a unique molecular identifier (UMI);
(c) amplifying and sequencing cDNA transcribed from the target gene, to determine the identity of the edited base, on a cell-by-cell basis;
(d) performing gene expression analysis on a cell-by-cell basis using short-read sequencing; and
(e) comparing the results of (c) and (d) for each cell, to determine how the edited base alters gene expression.

2. The method of claim 1, wherein (c) is done by long-range sequencing, the long-read sequencing comprises single molecule real time (SMRT) sequencing or nanopore sequencing.

3. The method of claim 1, wherein (b) is done by encapsulating each cell in a droplets and creating the cDNA in the droplets.

4. The method of any prior claim, wherein (d) is done by short range sequencing.

5. The method of claim 1, comprising contacting the genetically modified cells with a drug candidate to determine whether the candidate reverses any changes in gene expression that are caused by the edited base.

6. The method of claim 1, wherein the cells are mammalian cells.

7. The method of claim 1, wherein the cells are blood cells.

8. The method of claim 1, wherein the cells are cultured cells.

9. The method of claim 1, wherein the cells are exposed to a single base editor in (a).

10. The method of claim 1, wherein the cells are exposed to multiple base editors in (a).

11. The method of claim 1, wherein the method comprises making cDNA from the cells in droplets to make cDNA, and
specifically amplifying the target gene by PCR from the cDNA, sequencing the PCR products by long range sequencing, and then analyzing the long range sequence reads to determine the identity of the edited base in the cells on a cell-by-cell basis; and
sequencing the remainder of the cDNA by short-range sequencing, and then analyzing the short range sequence reads to determine a gene expression profile for the cells on a cell-by-cell basis.

12. The method of claim 1, wherein the short-range sequencing uses reversible terminators.

13. The method of claim 11, wherein the droplets contain beads.

14. The method of claim 1, wherein the base editing is done by a CRISPR-based editor.

15. The method of claim 1, where step (e) is done by matching data obtained from (c) that is associated with a barcode with data obtained from (d) that is associated with the same barcode.
